(19) United States
(12) Patent Application Publication
(10) Pub. No.: US 2022/0361902 A1
(43) Pub. Date: Nov. 17, 2022

(54) APPARATUSES AND METHODS FOR SETTING POSITION OF A SHEATH IN A SCOPE

(71) Applicants: Jean-Martin Baillargeon, Seattle, WA (US); Hugo X. Gonzalez, Woodville, WA (US)

(72) Inventors: Jean-Martin Baillargeon, Seattle, WA (US); Hugo X. Gonzalez, Woodville, WA (US)

(21) Appl. No.: 17/663,292

(22) Filed: May 13, 2022

Related U.S. Application Data
(60) Provisional application No. 63/259,902, filed on May 14, 2021.

(51) Int. Cl. A61B 17/22

(52) U.S. Cl. CPC A61B 17/22012; A61B 2017/00469

(57) ABSTRACT

Devices and methods for setting position of a medical tool within an endoscope. In an illustrative embodiment, a device includes a housing that is configured to be connectable to a distal end of a scope and a sheath stop component that is configured to limit distal movement of a sheath received within the scope.

### CROSS-REFERENCE TO RELATED APPLICATION

This application claims the benefit of priority to U.S. Provisional Patent Application Ser. No. 63/259,902, filed May 14, 2021, the contents of which are incorporated herein by reference in their entirety.

### BACKGROUND

Medical tools as used in endoscopes typically are inserted into the scope before the scope is inserted into the patient. The position of the tool or a sheath of the tool is typically precisely set to ensure consistent interaction with tissue. In one technique, an operator places their finger over the working channel port of the scope, then the tool and tool sheath is advanced until contact is made with the operator's finger.

### SUMMARY

Various disclosed embodiments include illustrative devices and methods for setting position of a medical tool within an endoscope.

In an illustrative embodiment, a method includes connecting a distal end of a scope to a sheath distance setting device, inserting a sheath into the scope until the sheath makes contact with the sheath distance setting device, and removing the distal end of the scope from the sheath distance setting device.

In another illustrative embodiment, a device includes a housing that is configured to be connectable to a distal end of a scope and a sheath stop component that is configured to limit distal movement of a sheath received within the scope.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 is a side view of an endoscope with an inserted medical tool;

FIG. 2 is a perspective view of the distal end of the endoscope of FIG. 1;

FIG. 3 is a perspective view of a sheath setting jig used with the endoscope of FIG. 1;

FIG. 4 is a side view of the sheath setting jig of FIG. 3 mounted to the endoscope of FIG. 1;

FIG. 5 is a perspective view of a sheath setting jig used with the endoscope of FIG. 1;

FIG. 6 is a cross-sectional view of the sheath setting jig of FIGS. 5 with the endoscope of FIG. 1 received therein; and

FIG. 7 is a flow diagram of an exemplary process for setting sheath position.

### DETAILED DESCRIPTION

The following description is merely illustrative in nature and is not intended to limit the present disclosure, application, or uses. The following description explains, by way of illustration only and not of limitation, various embodiments of devices and methods for setting tool sheath positioning within an endoscope.

Various embodiments of an endobronchial ultrasound (EBUS) system and its related components and parts will now be described with reference to the accompanying figures. The terminology used in the description presented herein is not intended to be interpreted in any limited or restricted manner. Rather, the terminology is simply being utilized in conjunction with a detailed description of embodiments of the devices, methods, and related components. Furthermore, embodiments may comprise several novel features, no single one of which is solely responsible for its desirable attributes or is believed to be essential to practicing the disclosure herein described.

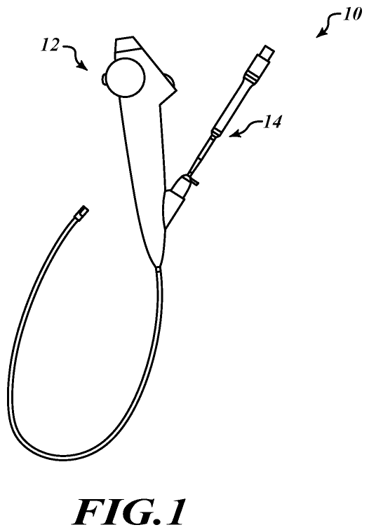

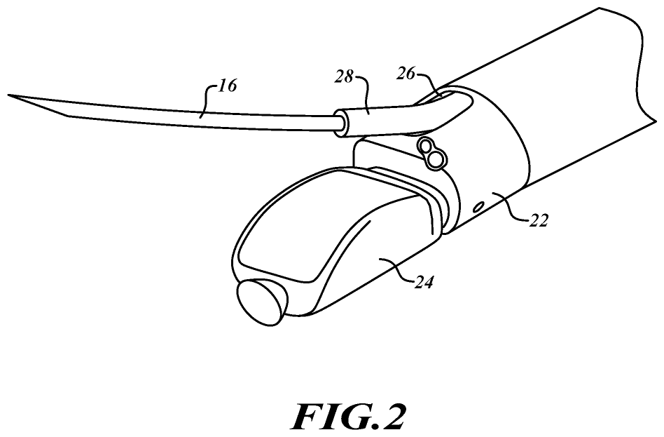

Referring now to FIGS. 1 and 2, a system 10 includes a medical tool 14 receivable within an endoscope 12. The medical tool 14 may include a needle 16 that is extendable at an angle from an exit port 26 at a distal end 22 of the endoscope 12. The needle 16 is slidable within a sheath 28. The sheath 28 is slidably received within a working channel of the endoscope 12.

In various embodiments, the medical tool 14 may be any medical device receivable within the endoscope 12, such as, without limitation, a needle, a cytology brush, a clamp, or comparable devices.

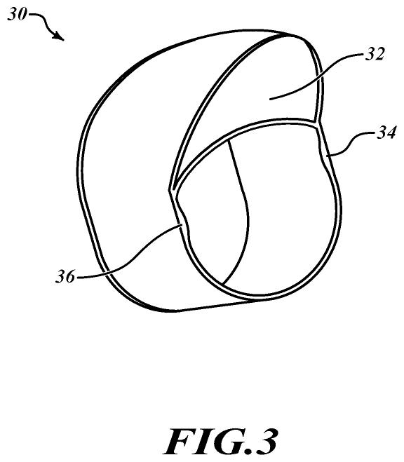

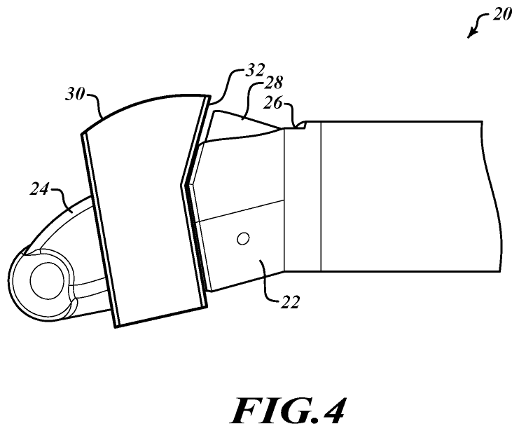

Referring additionally to FIGS. 3 and 4, in various embodiments, a sheath distance setting jig 30 may be a hollow structure. A proximal opening of the sheath distance setting jig 30 is partially blocked by a datum surface 32. The sheath distance setting jig 30 is receivable over a portion of the distal end 22 of the endoscope 12. The sheath distance setting jig 30 is configured to mount over a transducer section 24 of the distal end 22 of the endoscope 12. With the sheath distance setting jig 30 mounted to the distal end 22 of the endoscope 12, an operator inserts the sheath 28 of the medical tool 14 through a working channel of the endoscope 12 and out of the exit port 26. The datum surface 32 is configured to stop or provide feedback as the sheath 28 exits the exit port 26. Once the operator of the sheath 28 feels the sheath 28 make contact with the datum surface 32, the operator stops advancing the sheath 28 and may at that point lock the longitudinal position of the sheath 28 to the endoscope 12 by engaging a locking mechanism at a handle (FIG. 1). Sheath to handle locking mechanisms are well known to those skilled in the art.

In various embodiments, the sheath distance setting jig 30 may include snap fit features, such as, without limitation, tabs 34 and 36. The sheath distance setting jig 30 is configured to be flexible at least in a width dimension, such that the tabs 34 and 36 and the wall connected thereto are able to flex outwardly until the tabs 34 and 36 are received within a relief section located proximal from the transducer section 24. When the sheath distance setting jig 30 is in a resting configuration, an internal dimension between the side tabs 34 and 36 is smaller than a cross-sectional width of the transducer section 24 but larger than a cross-sectional dimension of a position just proximal of the transducer section 24. The tabs 34 and 36 maintain the sheath distance setting jig 30 in a desirable longitudinal position such that the datum surface 32 is located a predefined distance from the exit port 26. In various embodiments, a normal vector of the datum surface 32 is approximately parallel to a longitudinal axis of the exit port 26, which includes an exit ramp (not shown). The flexibility of the tabs 34 and 36 and the sheath distance setting jig 30 are configured to resist expansion in response to a distal force provided by the advancing sheath 28. Once the position of the sheath 28 has been set, the sheath distance setting jig 30 is removed. The sheath distance setting jig 30 may be removed by an operator pulling the sheath distance setting jig 30 distally to overcome the attachment force applied by the sheath distance setting jig 30 and the tabs 34 and 36. The sheath distance setting jig 30 may also be removed by the operator applying a distal force in conjunction with the operator pinching opposing sides of the sheath distance setting jig 30 thus causing the sides of the sheath distance setting jig 30 with the tabs 34 and 36 to expand laterally, thereby allowing for removal. The expansion of the tabs 34 and 36 may produce a width dimension greater than the width of the transducer section 24.

In various embodiments, the sheath distance setting jig 30 may be created using various materials, such as, without limitation, medical grade metals or plastics or comparable materials. Various techniques may be used for creating the sheath distance setting jig 30, such as, without limitation, 3D printing, molding, laser welding, or comparable techniques. The sheath distance setting jig 30 may include a cross-hatched frame with the membrane formed over the frame.

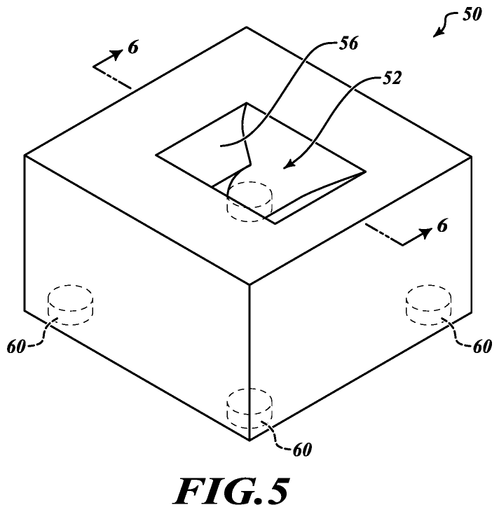

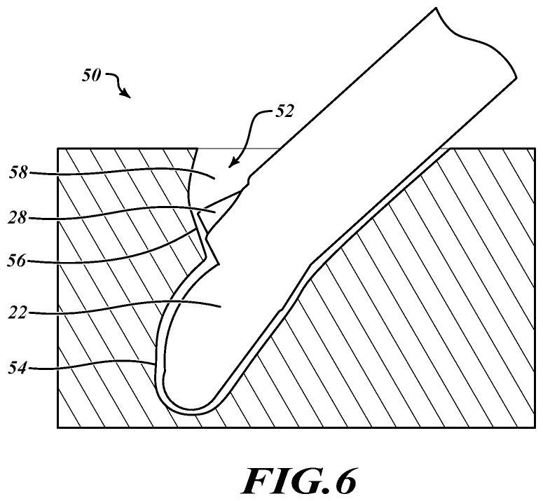

Referring now to FIGS. 5 and 6, a jig 50 is configured to rest on a tabletop (not shown) with stabilizing features 60, such as rubber feet, suction devices, or comparable devices. The jig 50 is a block-like structure that includes an opening 52 at a top surface to a cavity. At a lower portion of the cavity is a transducer head section 54 configured to receive the transducer section 24 of the endoscope 12. Above the transducer head section 54 within the cavity is a datum section 58 that includes a datum face 56. The location of the datum face 56 within the datum section 58 are configured to limit how far the sheath 28 can extend outside of the exit port 26. After the jig 50 receives the distal end 22 of the endoscope 12, the sheath 28 is advanced through the working channel of the endoscope 12 until the sheath 28 leaves the exit port 26 and makes contact with a datum face 56 within the jig 50. Once the sheath 28 makes contact with a datum face 56, the sheath 28 is locked to the handle. The endoscope 12 with the locked sheath 28 is now ready for insertion into a patient.

In various embodiments, the jig 50 may be created using various materials, such as, without limitation, medical grade metals or plastics or comparable materials. Various techniques may be used for creating the jig 50, such as without limitation, 3D printing, molding, machining, chemical etching, laser welding, or comparable techniques. The jig 50 may be created as two halves that are combined with a latch, adhesive, or comparable materials.

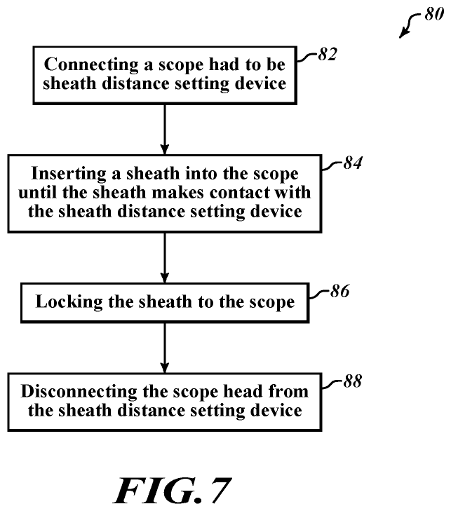

FIG. 7 shows a flowchart of a method 80 for setting sheath depth in an endoscope. At a block 82, a distal end of a scope is connected to a sheath distance setting device. At a block 84, a sheath is inserted into the scope until the sheath makes contact with the sheath distance setting device. At a block 86, the sheath is locked to the scope. At a block 88, the distal end of the scope is removed from the sheath distance setting device.

In some embodiments, connecting includes sliding the sheath distance setting device over a transducer head of the distal end of the scope.

In some embodiments, sliding includes snap fitting the distance setting device to the distal end of the scope.

In some embodiments, connecting includes placing a sheath advancement stopping device at a predefined distance from a working channel exit port of the scope.

In some embodiments, inserting includes inserting until a distal edge of the sheath makes contact with the sheath advancement stopping device.

In some embodiments, connecting includes inserting the distal end of the scope into a sheath setting jig.

In some embodiments, inserting the distal end of the scope into the sheath setting jig includes inserting an ultrasound transducer housing of the scope into an ultrasound transducer cavity of the jig and receiving a sheath stopping feature of the jig at a predefined distance from a working channel exit port of the scope.

In some embodiments, the sheath setting jig rests on a stable surface.

The description of the invention is merely exemplary in nature and variations that do not depart from the gist of the invention are intended to be within the scope of the invention. Such variations are not to be regarded as a departure from the spirit and scope of the invention.

### EMBODIMENTS

A. A method comprising connecting a distal end of a scope to a sheath distance setting device; inserting a sheath into the scope until the sheath makes contact with the sheath distance setting device; and removing the distal end of the scope from the sheath distance setting device.

B. The method of A, further comprising: locking the sheath to the scope before removing the distal end of the scope from the sheath distance setting device.

C. The method of A, wherein connecting includes sliding the sheath distance setting device over a transducer head of the distal end of the scope.

D. The method of C, wherein sliding includes snap fitting the sheath distance setting device to the distal end of the scope.

E. The method of C, wherein connecting includes placing a sheath advancement stopping surface of the sheath distance setting device at a predefined distance from a working channel exit port of the scope.

F. The method of E, wherein inserting includes inserting the sheath until a distal edge of the sheath makes contact with the sheath advancement stopping surface.

G. The method of A, wherein connecting includes inserting the distal end of the scope into the sheath distance setting device.

H. The method of G, wherein inserting the distal end of the scope into the sheath distance setting device includes: inserting an ultrasound transducer housing of the scope into an ultrasound transducer cavity of the sheath distance setting device; and receiving a sheath stopping feature of the sheath distance setting device at a predefined distance from a working channel exit port of the scope.

I. The method of G, further comprising stabilizing the sheath distance setting device on a stable surface.

J. A device comprising: a housing configured to be connectable to a distal end of a scope; and a sheath stop component configured to limit distal movement of a sheath received within the scope.

K. The device of J, wherein the housing is further configured to be slidably received over a transducer head of the distal end of the scope.

L. The device of K, wherein the housing comprises: a framed configured to form a hollow passage; and one or more tabs configured to protrude from the frame into the hollow passage.

M. The device of L, wherein at least one of the frame or the one or more tabs are configured to snap fit onto the distal end of the scope.

N. The device of K, wherein the sheath stop component is disposed at a predefined distance from a working channel exit port of the scope when the housing is connected to the distal end of the scope.

O. The device of J, wherein: the housing further comprises a cavity configured to receive the distal end of the scope; and the sheath stop component is further configured to be disposed at a predefined distance from a working channel exit port of the scope when the distal end of the scope is received within the cavity.

P. The device of O, further comprising a stabilization component attachable to a bottom surface of the housing, the stabilization component configured to maintain stability of the housing on a surface.

Q. The device of O, wherein a normal vector of the sheath stop component is parallel to a longitudinal axis of a ramp of the working channel exit port of the scope.

Although the preferable embodiments of the present invention have been described hitherto, the present invention is not limited to these embodiments. Additions, omissions, substitutions, and other modifications can be made without departing from the spirit or scope of the present invention.

What is claimed is:

1. A method comprising:
connecting a distal end of a scope to a sheath distance setting device;
inserting a sheath into the scope until the sheath makes contact with the sheath distance setting device; and
removing the distal end of the scope from the sheath distance setting device.

2. The method of claim 1, further comprising:
locking the sheath to the scope before removing the distal end of the scope from the sheath distance setting device.

3. The method of claim 1, wherein connecting includes:
sliding the sheath distance setting device over a transducer head of the distal end of the scope.

4. The method of claim 3, wherein sliding includes:
snap fitting the sheath distance setting device to the distal end of the scope.

5. The method of claim 3, wherein connecting includes:
placing a sheath advancement stopping surface of the sheath distance setting device at a predefined distance from a working channel exit port of the scope.

6. The method of claim 5, wherein inserting includes:
inserting the sheath until a distal edge of the sheath makes contact with the sheath advancement stopping surface.

7. The method of claim 1, wherein connecting includes:
inserting the distal end of the scope into the sheath distance setting device.

8. The method of claim 7, wherein inserting the distal end of the scope into the sheath distance setting device includes:
inserting an ultrasound transducer housing of the scope into an ultrasound transducer cavity of the sheath distance setting device; and
receiving a sheath stopping feature of the sheath distance setting device at a predefined distance from a working channel exit port of the scope.

9. The method of claim 7, further comprising stabilizing the sheath distance setting device on a stable surface.

10. A device comprising:
a housing configured to be connectable to a distal end of a scope; and
a sheath stop component configured to limit distal movement of a sheath received within the scope.

11. The device of claim 10, wherein the housing is further configured to be slidably received over a transducer head of the distal end of the scope.

12. The device of claim 11, wherein the housing comprises:
a framed configured to form a hollow passage; and
one or more tabs configured to protrude from the frame into the hollow passage.

13. The device of claim 12, wherein at least one of the frame or the one or more tabs are configured to snap fit onto the distal end of the scope.

14. The device of claim 11, wherein the sheath stop component is disposed at a predefined distance from a working channel exit port of the scope when the housing is connected to the distal end of the scope.

15. The device of claim 10, wherein:
the housing further comprises a cavity configured to receive the distal end of the scope; and
the sheath stop component is further configured to be disposed at a predefined distance from a working channel exit port of the scope when the distal end of the scope is received within the cavity.

16. The device of claim 15, further comprising a stabilization component attachable to a bottom surface of the housing, the stabilization component configured to maintain stability of the housing on a surface.

17. The device of claim 15, wherein a normal vector of the sheath stop component is parallel to a longitudinal axis of a ramp of the working channel exit port of the scope.
